(19) United States
(12) Patent Application Publication
(10) Pub. No.: US 2004/0267122 A1
(43) Pub. Date: Dec. 30, 2004

(54) Medical image user interface

(76) Inventors: Desikachari Nadadur, Issaquah, WA (US); David E. Gustafson, North Bend, WA (US)

Correspondence Address: Siemens Corporation, Attn: Elsa Keller, Legal Administration, Intellectual Property Department, 170 Wood Avenue South, Iselin, NJ, 08830, US

(21) Appl. No.: 10/608,284

(22) Filed: Jun. 27, 2003

(51) Int. Cl. A61B 8/00

(52) U.S. Cl. 600/440

(57) ABSTRACT

In one embodiment, a user interface is provided comprising at least three display areas that are simultaneously displayed. The first display area displays a medical image, the second display area displays a plurality of image frames of the medical image, and the third display area displays a data plot. In another embodiment, a user interface is provided comprising at least two display areas that are simultaneously displayed. The first display area displays a medical image, and the second display area displays a plurality of image frames of the medical image. In response to receiving a selection of an image frame in the second display area, the selected image frame is displayed in the first display area. Other embodiments are provided, and each of the embodiments described herein can be used alone or in combination with one another.

### BACKGROUND

Diagnostic medical imaging systems are used to image various body tissues and organs to view the internal conditions of a patient. An acquired or recalled medical image can be displayed on a display device of an image acquisition system, such as an ultrasound imaging system, or an image review system, such as a workstation. The medical image can be displayed by itself or in combination with data plots associated with the image. For example, in an ultrasound cardiac application, an ultrasound image of the heart is often displayed with an ECG waveform. Although existing medical image user interfaces provide valuable information, there is a need for an improved medical image user interface.

### SUMMARY

The present invention is defined by the following claims, and nothing in this section should be taken as a limitation on those claims.

By way of introduction, the embodiments described below relate to a medical image user interface. In one embodiment, a user interface is provided comprising at least three display areas that are simultaneously displayed. The first display area displays a medical image, the second display area displays a plurality of image frames of the medical image, and the third display area displays a data plot. In another embodiment, a user interface is provided comprising at least two display areas that are simultaneously displayed. The first display area displays a medical image, and the second display area displays a plurality of image frames of the medical image. In response to receiving a selection of an image frame in the second display area, the selected image frame is displayed in the first display area. Other embodiments are provided, and each of the embodiments described herein can be used alone or in combination with one another.

The embodiments will now be described with reference to the attached drawings.

### BRIEF DESCRIPTION OF THE DRAWINGS

FIG. 1 is an illustration of a medical image user interface of an embodiment.

FIG. 2 is a flow chart of a method of using a medical image user interface of an embodiment.

FIG. 3 is a block diagram of a medical diagnostic ultrasound imaging system that can be used to implement the medical image user interface of an embodiment.

### DETAILED DESCRIPTION OF THE PRESENTLY PREFERRED EMBODIMENTS

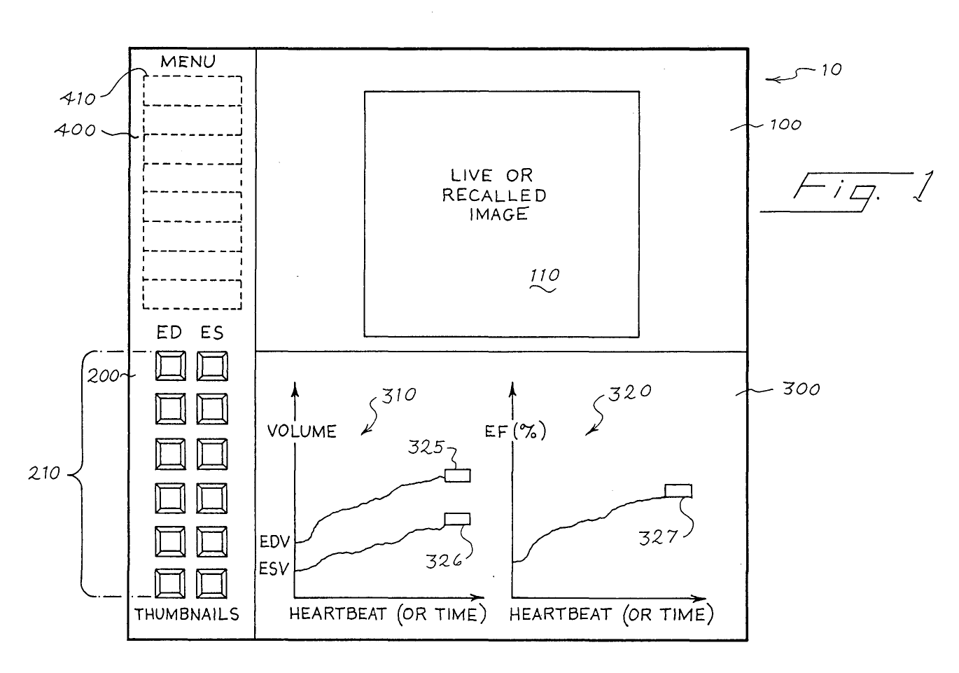

Turning now to the drawings, FIG. 1 is an illustration of a medical image graphic user interface (GUI) 10 of an embodiment. As shown in FIG. 1, the user interface 10 comprises four display areas 100, 200, 300, 400. As used herein, a “display area” can be an individual window/pane or a portion of an individual window/pane. Accordingly, two or more display areas can be part of a single window, or each can be a separate window. As shown in FIG. 1, the display areas 100, 200, 300, 400 are simultaneously displayed. As used herein, display areas are “simultaneously displayed” when they are displayed together at a given time. “Simultaneously displayed” does not necessarily mean that the contents of the display areas are updated exactly at the same time. Simultaneously displayed display areas can be displayed on a single or multiple display devices (e.g., the first display area can be displayed on a first display device, while the second and third display areas are displayed on a second display device).

In one embodiment, the first display area 100 displays a medical image 110, and the second display area 200 displays a plurality of image frames 210 of the medical image 110. As shown in FIG. 1, the size of the image frames 210 in the second display area 200 is smaller that the size of the medical image 110 displayed in the first display area 100. For example, the plurality of images 210 in the second display area 200 can be “thumbnail” versions of a “full-size” image 110 displayed in the first display area 100. (In other embodiments, the image frames in the second display area 200 can be the same size as the medical image 110.) As described below, the image 110 displayed in the first display area 100 can be a live (real-time) acquisition image or a recalled image. Additionally, the medical image 110 can be either a still image frame or a sequence of images frames (i.e., a CINE clip). The third display area 300 displays one or more data plots (such as data plots 310, 320) associated with the displayed images. The fourth display area 400 displays a menu 410, which can display various user-selectable features (not shown).

The placement of one or more of the display areas 100, 200, 300, 400 can be fixed or user configurable. For example, the placement of the first display area 100 can be determined in the system specification of the device implementing the user interface 10, while the placement of the second, third, and fourth display areas 200, 300, 400 can be user configurable, thereby introducing flexibility into the display. User configurability can be facilitated by a simple drag-and-drop operation. Once satisfied, the user can lock the display areas into position to prevent accidentally dragging and dropping them to different places. Further, if the user desires, he can turn off or hide any of the display areas 100, 200, 300, 400 and expand the other display areas. For example, the user can turn off the third display area 300 to hide the plots 310, 320 if he wishes only to look at the first display area 100 and then enlarge the first display area 100 into the space formerly occupied by the third display area 300, keeping the aspect ratio and the image resolution intact so the image 110 can be easily seen. Similar adjustments can be made to any of the other display areas. Further, the expansion of a given display area can be preset or user configurable. For example, a user may want to turn off the third display area 300 and expand the second display area 200 into this space to see more thumbnails. The user can also hide the live or recalled image 110 in the first display area 100 and expand the third display area 300 into the space of the first display area 100 to see the plots 310, 320 more clearly. Further, controls can be provided to allow a user to enlarge, scale, zoom, or pan the image 110 or the plots 310, 320.

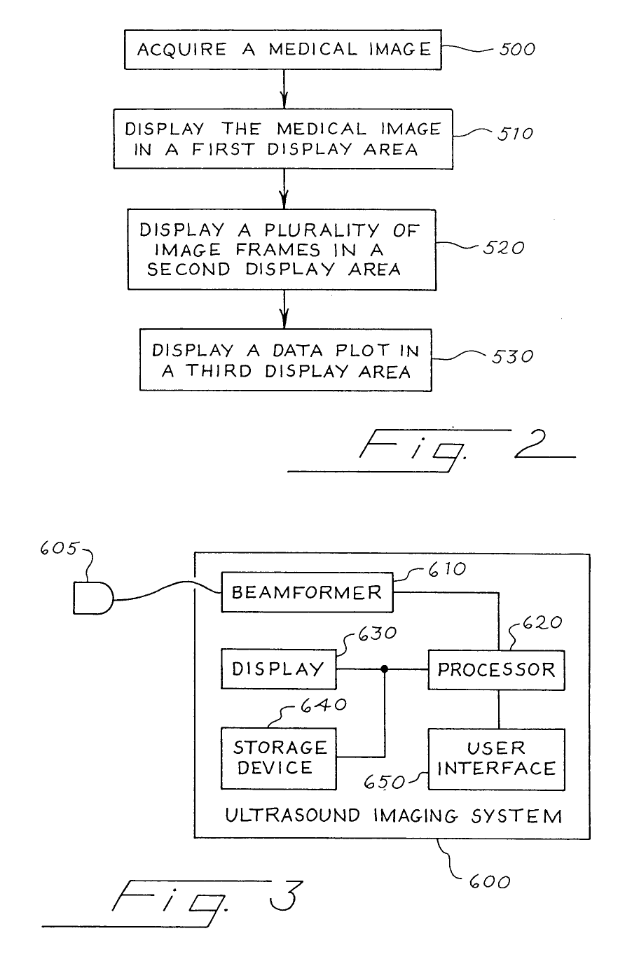

To illustrate the operation of the user interface 10, the user interface 10 will be described in conjunction with a cardiac ultrasound imaging application to provide dynamic, intuitive, and efficient display of automated quantification results. It should be noted that the user interface 10 can be used to image other organs and that other types of medical images can be used. Turning again to the drawings, FIG. 2 is a flow chart that will be used to illustrate the operation of the user interface 10 shown in FIG. 1 during live image acquisition. As shown in FIG. 2, an ultrasound image is acquired with a medical diagnostic ultrasound imaging system (act 500). An exemplary ultrasound system 600 is shown in FIG. 3. This ultrasound system 600 comprises a transducer probe 605, a beamformer 610, a processor 620, a display device 630 (which displays the graphical user interface 10), a storage device 640, and a user interface 650, such as a keyboard, trackball, mouse, etc. The ultrasound system 600 can comprise additional components (such as additional display devices), which are not shown in FIG. 6 for simplicity. During an ultrasound examination, a sonographer contacts the transducer probe 605 with a patient, and the ultrasound system 600 generates an ultrasound image. In general, the ultrasound system's processor 620 causes the beamformer 610 to apply a voltage to the transducer 605 to cause it to vibrate and emit an ultrasonic beam into the portion of the patient's body in contact with the transducer 605. Ultrasonic energy reflected from the patient's body impinges on the transducer 605, and the resulting voltages created by the transducer 605 are received by the beamformer 610. The processor 620 processes the sensed voltages to create an ultrasound image frame.

Turning back to FIG. 2, the created medical image 110 is displayed in the first display area 100 (act 510). The medical image 110 comprises image frames generated by the ultrasound system 600. These images are displayed in the first display area 100 in rapid succession as they are being generated, with a newly-generated image frame replacing a currently-displayed image frame, to provide a display of a moving image (in this example, of a beating heart). A plurality of image frames 210 of the medical image 110 are displayed in the second display area 200 (act 520). In this embodiment, the plurality of image frames 210 are captured as the medical image is being acquired. Specifically, the plurality of image frames 210 comprises thumbnail images 210 of end-diastolic (ED) and end-systolic (ES) frames for each heartbeat. To capture these images, the ultrasound system 600 can use R-wave tagging information to identify the ED frame along with using well-published equations for identifying the ES frame.

In one embodiment, the thumbnails 210 are paginated, with each page containing five or six pairs (ED & ES) of thumbnails. The number of thumbnails in each page can be user configurable. This number determines the size of the thumbnails and, hence, the details that appear in them. In this embodiment, if the thumbnails exceed a preset number, the user interface 10 displays a new page and shows the new thumbnails being created. Additionally, a scrollbar appears (e.g., on the right side of the second display area 200 between the second display area 200 and the third display area 300) to allow the user to navigate to previous or next pages. In this embodiment, the user can scroll to previous pages of thumbnails even when the ultrasound system 600 is in an acquisition mode. Other mechanisms, such as navigation buttons, may be provided to allow the user to navigate through the pages.

As the medical image 110 is being displayed in the first display area 100 and the plurality of image frames 210 are being displayed in the second display area 200, one or more data plots are displayed in the third display area 300 (act 530). In this embodiment, two data plots 310, 320 are displayed. The first data plot 310 is a graph of end-diastolic (ED) and end-systolic (ES) volume versus heartbeat (or time), and the second data plot 320 is a graph of ejection fraction versus heartbeat (or time). It should be noted that other types of information can be shown in the data plots 310, 320, such as stroke volume, stroke index, cardiac output, cardiac index or any data that varies over time or heartbeat. Optionally, average values may be computed and displayed for values plotted over time. The indicators 325, 326, 327 on the plots 310, 320 show the current time point or heartbeat number currently being processed (i.e., where the image frame displayed in the first display region 100 is along the heartbeat (or time) axis). Although the indicators 325, 326, 327 are shown as breaks in the plots, other types of indicators can be used, such as, but not limited to, a bright point, a vertical line, or any other symbol. Scroll bars may be provided to allow a user to scroll back to desired points on the plots 310, 320. Although not show, the user interface 10 can also facilitate the display of an ECG waveform.

In this embodiment, the user interface 10 allows a user to alter the display in the various display areas 100, 200, 300. In general, the user can suspend (or “freeze”) the acquisition process and alter the display in one or more of the display areas 100, 200, 300. Preferably, a “single-click” operation is used to both freeze the acquisition process and alter the display. Alternatively, multiple actions can be required, such as first depressing a control panel button (or single clicking) to freeze the acquisition process and then making a selection in one of the display areas 100, 200, 300 (e.g., by double clicking). The following paragraphs illustrate examples in which a user makes a selection in the first, second, and third display areas 100, 200, 300. For simplicity, a single-click operation of a mouse will be used to illustrate the examples. It should be noted that any type of user interface device can be used, and multiple actions (instead of a single-click) can be required to implement the functionality.

When a user clicks on the medical image 110 in the first display area 100, the system 600 enters the freeze mode. In the freeze mode, the currently-displayed image frame of the medical image 110 remains displayed in the first display area 100. When in the freeze mode, the user can select a pair of corresponding or separate ED and ES frames to save to a report. Preferably, reduced-size images with borders overlaid are saved to the report along with the corresponding numeric results and the portions of the plots 310, 320 between the chosen images.

When a user clicks on one of the image frames shown in the second display area 200 (i.e., one of the thumbnail ED or ES frames), the system 600 enters the freeze mode and the selected image is displayed in the first display area 100 in a larger size than is shown in the second display area 200. In this way, the thumbnails of the live acquisition stream act as bookmarks to help the user easily and quickly access ED and ES frames. Additionally, the indicators 325, 326, 327 in the data plots 310, 320 are moved to points in the plots 310, 320 that correspond to when the selected image frame was generated.

When a user clicks on a point in one of the plots 310, 320, the system 600 enters the freeze mode and moves the indicators 325, 326, 327 to the selected point. Additionally, the first display area 100 displays the closest image frame corresponding to the selected point (if one of the axes is time) or the ED frame corresponding to the selected point (if one of the axes is heartbeat). Further, the second display area 200 is scrolled to the ED frame thumbnail corresponding to the heartbeat where the selected point is located.

Regardless of which display area was selected to enter the freeze mode, a user can playback the medical image 110 with all or some of its frames over some specified interval. Additionally, a user can unfreeze the system 600 and resume the acquisition process by clicking on the medical image 100. When the resumption happens, the cursors 325, 326, 327 on the plots 310, 320 jump forward to the end of the plots 310, 320 (i.e., to the current time point), current acquisition images are shown in the first display area 100, and the thumbnails page jumps to the most recent page to start creating the thumbnails in the second display area 200. A marker (not shown) can be placed on the plots 310, 320 to indicate where the user paused/resumed the acquisition process. Preferably, scrolling is allowed in the first display area 100 and the third display area 300 only when the system 600 enters suspend mode and not when the system 600 is in acquisition mode.

There are several alternatives that can be used with these embodiments. For example, instead of displaying all three display areas, a user interface can display the first and second display areas without the third display area. In this way, the user interface can be used to provide bookmarks in the second display area to specific image frames of the medical image shown in the first display area. In another embodiment, the user interface 10 is used in a recalled image mode, instead of in a live acquisition mode as in the embodiment described above. In operation, the ultrasound system 600 stores a generated ultrasound image in digital form in the storage device 640, on removable media (e.g., a magneto-optical disk), or in a component connected to a network (e.g., a local area network in a hospital or the Internet). When the user enters a recalled image mode (e.g., to see a previously-stored clip for reference during the current acquisition), the recalled image (instead of a live image) is displayed in the first display area 100. The operation of the user interface in the recalled image mode is similar to that in the live acquisition mode.

In the embodiments discussed above, the user interface was implemented on a medical imaging device for online and offline processing of images. In another embodiment, the user interface is implemented on a device other than an image acquisition system to allow a user to view recalled images when performing offline processing of previously acquired image data (image clips). Such devices are referred to herein as “image review systems.” An image review system can be any device other than the medical imaging system that created the image being reviewed. An image review system can be, for example, a general-purpose or specialized computer (e.g., a desktop computer in the user's office/home), a personal digital assistant (PDA), or another medical imaging system. The medical images and associated data can be provided from a medical imaging system to the image review system via removable media (e.g., a magneto-optical disk), a network (e.g., a local area network in a hospital or the Internet), a wireless transmission, or any other suitable technique. The image review system can perform various functions, such as displaying images, making measurements of anatomical structures and other features shown in the images, and creating medical reports.

Regardless of where it resides (e.g., in an imaging system or a review system), any suitable hardware and/or software components can be used to implement the user interface. For example, the software (i.e., computer-readable program code) for the user interface can be stored in the storage device 640 of the ultrasound system 600 and executed by the processor 620. Although only a single processor 620 is shown in FIG. 3, it should be understood that the ultrasound system 600 can comprise multiple processors and that the functionality described herein can be performed by a single processor or can be distributed among several processors. Alternatively, some or all of the functionality described herein can be implemented purely with hardware. For simplicity, the term “processor” is being used in the claims to refer to the hardware and/or software components that are used to implement the user interface. In addition, the functionality described herein can be implemented in software using multi-threading.

Finally, as described above, the user interface can be used with medical images other than cardiac or ultrasound images. When used in other applications, some of the labels and techniques used in the above example can be replaced with labels and techniques appropriate for those applications. For example, the ED and ES labeling of the thumbnails can be replaced with labels appropriate for the organ being imaged or the labels can be removed altogether. Further, thumbnails can be placed at pre-specified time intervals instead of using heartbeats as in cardiac imaging. Also, the plots in the third display area can be of different types of variables and are not restricted to the two shown in FIG. 1. These variables can be factory preset depending on the application or can be user configurable. Further, the user interface 10 can be extended to three- and four-dimensional imaging and quantification and can be used not only with B-mode images, but also with color mode, Doppler, and other modes and combinations thereof. More generally, any type of medical image, not merely an ultrasound image can be used with these embodiments, and the claims should not be limited to any particular type of medical image unless explicitly recited therein. Examples of different types of medical images that can be used with these embodiments include, but are not limited to, images created with any of the following imaging modalities: computed tomography (CT), magnetic resonance imaging (MRI), computed radiography, magnetic resonance, angioscopy, color flow Doppler, cystoscopy, diaphanography, echocardiography, fluoresosin angiography, laparoscopy, magnetic resonance angiography, positron emission tomography, single-photon emission computed tomography, x-ray angiography, computed tomography, nuclear medicine, biomagnetic imaging, culposcopy, duplex Doppler, digital microscopy, endoscopy, fundoscopy, laser surface scan, magnetic resonance spectroscopy, radiographic imaging, thermography, and radio fluroscopy.

It is intended that the foregoing detailed description be understood as an illustration of selected forms that the invention can take and not as a definition of the invention. It is only the following claims, including all equivalents, that are intended to define the scope of this invention.

What is claimed is:

1. A method for displaying a medical image, the method comprising:
displaying a medical image in a first display area;
displaying a plurality of image frames of the medical image in a second display area; and
displaying a data plot in a third display area;
wherein the first, second, and third display areas are simultaneously displayed.

2. The method of claim 1, wherein a size of the image frames displayed in the second display area is smaller than a size of the medical image displayed in the first display area.

3. The method of claim 1 further comprising:
(a) receiving a user selection in one of the first, second, and third display areas; and
(b) altering a display of at least one of the first, second, and third display areas in response to the received user selection.

4. The method of claim 3, wherein (a) comprises receiving a selection of a point on the data plot in the third display area, and wherein (b) comprises:
displaying an indicator at the point selected on the data plot in the third display area;
displaying an image frame in the first display area corresponding to the point selected on the data plot; and
scrolling the plurality of image frames displayed in the second display area to the image frame corresponding to the point selected on the data plot.

5. The method of claim 3, wherein (a) comprises receiving a selection of an image frame displayed in the second display area, and wherein (b) comprises:
displaying an indicator at a point on the data plot in the third display area corresponding to the selected image frame; and
displaying the selected image frame in the first display area.

6. The method of claim 3, wherein (a) comprises receiving a selection of the medical image in the first display area, and wherein (b) comprises pausing the display of the medical image.

7. The method of claim 3, wherein the receiving of the user selection in (a) suspends a medical image acquisition operation.

8. The method of claim 3 further comprising, before (a), receiving a command to suspend a medical image acquisition operation.

9. The method of claim 1 further comprising:
displaying a menu in a fourth display area.

10. The method of claim 1, wherein the plurality of image frames comprises end-diastolic (ED) and end-systolic (ES) frames.

11. The method of claim 1, wherein the medical image comprises a live image.

12. The method of claim 1, wherein the medical image comprises a recalled image.

13. The method of claim 1, wherein the medical image comprises an ultrasound image.

14. The method of claim 1, wherein the data plot comprises a graph of at least one of the following: end-diastolic volume, end-systolic volume, ejection fraction, stroke volume, stroke index, cardiac output, and cardiac index.

15. The method of claim 1, wherein the first, second, and third display areas are displayed on a medical diagnostic image acquisition system.

16. The method of claim 1, wherein the first, second, and third display areas are displayed on an image review system.

17. A method for displaying a medical image, the method comprising:
simultaneously displaying a medical image in a first display area and a plurality of image frames of the medical image in a second display area;
receiving a selection of an image frame in the second display area; and
displaying the selected image frame in the first display area.

18. The method of claim 17, wherein a size of the image frames displayed in the second display area is smaller than a size of the medical image displayed in the first display area.

19. The method of claim 17 further comprising displaying a data plot in a third display area, wherein the third display area is simultaneously displayed with the first and second display areas.

20. The method of claim 19 further comprising displaying an indicator on the data plot corresponding to the selected image frame.

21. The method of claim 17, wherein the plurality of image frames comprises end-diastolic (ED) and end-systolic (ES) frames.

22. The method of claim 17, wherein the medical image comprises a live image.

23. The method of claim 17, wherein the medical image comprises a recalled image.

24. The method of claim 17, wherein the medical image comprises an ultrasound image.

25. The method of claim 17, wherein the first and second display areas are displayed on a medical diagnostic image acquisition system.

26. The method of claim 17, wherein the first and second display areas are displayed on an image review system.

27. A system for displaying a medical image, the system comprising:
at least one display device; and
a processor operative to:
simultaneously display a medical image in a first display area and a plurality of image frames of the medical image in a second display area on the at least one display device; and
in response to receiving a selection of an image frame in the second display area, display the selected image frame in the first display area.

28. The system of claim 27, wherein a size of the image frames displayed in the second display area is smaller than a size of the medical image displayed in the first display area.

29. The system of claim 27, wherein the processor is further operative to display a data plot in a third display area, wherein the first, second, and third display areas are simultaneously displayed.

30. The system of claim 29, wherein the process is further operative to display an indicator on the data plot corresponding to the selected image frame.

31. The system of claim 27, wherein the plurality of image frames comprises end-diastolic (ED) and end-systolic (ES) frames.

32. The system of claim 27, wherein the medical image comprises a live image.

33. The system of claim 27, wherein the medical image comprises a recalled image.

34. The system of claim 27, wherein the medical image comprises an ultrasound image.

35. The system of claim 27, wherein the at least one display device and processor are part of a medical diagnostic image acquisition system.

36. The system of claim 27, wherein the at least one display device and processor are part of an image review system.
